(19) United States
(12) Patent Application Publication
(10) Pub. No.: US 2002/0025320 A1
(43) Pub. Date: Feb. 28, 2002

(54) Sialidases as mucosal adjuvants

(76) Inventors: Prosper Boyaka, Birmingham, AL (US); Mariarosaria Marinaro, Bari (IT)

Correspondence Address: Glenna Hendricks, Esq., P.O. Box 2509, Fairfax, VA, 22031-2509, US

(21) Appl. No.: 09/887,530

(22) Filed: Jun. 25, 2001

Related U.S. Application Data
(63) Continuation-in-part of application No. 09/377,990, filed on Aug. 20, 1999.

(51) Int. Cl. A61K 39/00; A61K 38/47; A61L 9/04

(52) U.S. Cl. 424/184.1; 424/94.61; 424/46

(57) ABSTRACT

Organisms or proteins may be administered in conjunction with sialidases in a pharmaceutically acceptable carrier to heighten immune response. When sialidase is used in conjunction with an antibody to a CD43 molecule, the immune response-enhancing properties of the sialidases are further potentiated in a synergistic manner. The mucosal routes of administration include oral, nasal, rectal and vaginal routes.

This application is a continuation-in-part of U.S. application Ser. No. 09/377,990, filed Aug. 20, 1999, now pending, which takes priority from Provisional Patent Application 60/097,456, filed Aug. 21, 1998.

This invention was partly supported by a grants AI 18958, DK 44240, AI 35344, DE 90837 from the National Institutes of Health, United States Government. Hence, the United States Government has certain rights therein.

### FIELD OF THE INVENTION

This invention relates to use of bacterial sialidases as adjuvants for use in boosting immune response. Sialidases are particularly useful for enhancement of mucosal immune response. In a preferred embodiment, the sialidases are used in conjunction with antibodies against CD43 molecules.

### BACKGROUND OF THE INVENTION

The induction of immune responses in mucosal sites of the organism (i.e., intestinal, respiratory, and genito-urinary tracts) can be achieved by stimulation of immune cells in mucosal inductive sites (i.e., Peyer's patches, nasal and genito-urinary tract-associated lymphoreticular tissues) and by triggering migration of immune effector cells into systemic and mucosal compartments. Thus, while vaccines systemically administered can only stimulate systemic immunity, appropriate mucosal vaccines can induce both mucosal and systemic immune responses. However, mucosal administration of protein antigen often results in low immune response and may, in fact, cause mucosal (i.e., oral or nasal) tolerance. Further, conventional adjuvants such as aluminum hydroxide and Freud's complete adjuvant which display good adjuvanticity when parenterally administered have been found ineffective for purposes of mounting significant systemic immune responses when co-mucosally administered with protein antigens. Therefore, there is need for development of means for administration of mucosal vaccines which can provide effective immune protection at the portal of entry or at the site of reproduction of pathogenic organisms.

Sialidases or neuraminidases represent a group of enzymes produced by bacteria, viruses, parasites and mammalian cells which share the ability to remove terminal sialic acid residues from glycoproteins and glycolipids. The removal of cell surface sialic acids has a number of consequences for the functions and interactions of cells with other cells or other ligands. Sialic acids are the main contributors to the negative charge on cells and, in this regard, their removal favors interaction of cells with negatively charged ligands. For example, desialylation of T cells favor the cell's interaction with antigen presenting cells (APCs). It is believed this occurs because galactose residues and galactose receptors expressed by APCs have been unmasked by sialidase. It has also been shown that malignant transformation is associated with changes in cell surface sialic acid moieties and that desialylation of tumor cells enhances their recognition by cytotoxic T cells. As a consequence of desialylation of immune cells, a change in their migration (homing) toward organs occurs. Desialylation of T lymphocytes by intravenous injection of bacterial sialidases altered adhesive properties of T lymphocytes to high endothelial venules and enhanced their accumulation at sites of inflammation.

The leukocyte-specific surface molecule leukosialin (CD43) represents one of the most highly sialylated glycoproteins known on hemopoietic cells. It has been reported that CD43 cross-linkage increases the ability of T cell CD43 to interact with MHC class I molecules on antigen-presenting cells and enhances T cell activation.

Sialidases have been administered to humans. The sialidase from influenza virus is a well known antigen used to protect against influenza infection. In several attempts to enhance the immune responses to sialylated protein or to tumor cells, sialidases were administered parenterally with sialylated HIV protein or with tumor cells. A mixture of sialidase and galactose oxidase has also been administered parenterally to enhance the immune response to irrelevant protein antigens. In those studies, sialidase was used to increase the number of potential sites for galactose oxidase. The adjuvant effect observed was attributed to T cell activation by galactose oxidation. However, the use of sialidase as adjuvant for unrelated protein- or DNA-based vaccines and to promote mucosal immune responses was not previously known, though the conjugation molecule hemogglutinin-neuraminidase has been suggested.

### SUMMARY OF THE INVENTION

This invention provides means of enhancing immune responses systemically and mucosally. In the practice of the invention, organisms or proteins may be administered in conjunction with sialidases in a pharmaceutically acceptable carrier to heighten immune response. When sialidase is used in conjunction with an antibody to a CD43 molecule, the immune response-enhancing properties of the sialidases are further potentiated in a synergistic manner. The mucosal routes of administration include oral, nasal, rectal and vaginal routes.

### DETAILED DESCRIPTION OF THE INVENTION

The addition of sialidases to immunogenic compositions provides an improved means of enhancing immune response. The adjuvants of the invention are particularly useful in vaccines against pathogenic organisms when the mucosa is the site of invasion and/or toxic response and/or the site at which immunogenicity arises. Such infections include those whose port of entry is the mucosa of the respiratory, genito-urinary or alimentary tract such as those caused by, for example E. coli, Clostridium species, Salmonella species, and Shigella species whose target tissue is the alimentary tract or viral infections whose primary port of entrance is the respiratory tract.

It is interesting that the co-administration of a sialidase in conjunction with immunogenic toxins such as tetanus toxoid gave rise to systemic protection in the animal. When small amounts of antibodies against CD-43 were given in conjunction with sialidase, amounts of sialidase used to obtain desired results could be greatly reduced.

Target mucosa include those of the oral, nasal, rectal and vaginal mucosa. The compositions may be administered in various forms such as liquids or in the a dry form. However, other forms such as lotions, gels or solid supports with the antigen and adjuvants thereon may be useful.

Material and Methods

While mammalian sialidases are known, bacterial sialidases are more readily available, and may be conveniently used in the methods of the invention.

Bacterial sialidases used were Clostridium perfingens, Salmonella tvphimurium, and Arthrobacter urefaciens sialidases which were purchased from Sigma Chemical Co., St Louis, Mo. and Calbiochem Inc., Palo Alto, Calif. The anti-CD-43 antibody was obtained in the form of a clone designated S7 from Pharmingen Corporation, Palo Alto, Calif. The results disclosed herein have shown that intranasal administration of these sialidases with a protein antigen induced antigen-specific S-IgA Ab responses in mucosal secretions. Further, when tetanus toxoid was used as antigen, the adjuvant effect of sialidases mucosally co-admistered as adjuvant resulted in vaccine-specific serum Ab titers which were protective against a systemic challenge with tetanus toxin. Bacterial sialidases as adjuvant promoted mixed T helper type 1 and 2 (Th1 and Th2) responses. However IL-4 (Th2-type cytokine) was not induced consistent with the absence of IgE Ab responses.

When co-administered with anti-CD-43 antibody, lower doses (⅓ of optimal dose) of sialidase compared to amount used for optimal response without the antibody proved very efficacious.

Animal Studies

C57BL/6 mice were obtained from the Charles River Laboratories (Wilmington, Del.) and were maintained in the animal facility of the University of Alabama (UAB) Immunobiology Vaccine Center. Sterile food and water were provided ad libitum. Mice were pathogen free according to routine screening and biopsies of organs and were used at 8 5 to 12 weeks of age.

Mice were anesthetized and intranasally immunized with 30 μl (15 μl per nostril) of vaccine preparations on days 0, 7 and 14. Vaccines consisted on phosphate buffered saline (PBS) containing 20 μg of vaccine grade tetanus toxoid (TT; Connaught Laboratories, Inc., Swiftwater, Pa.) or 50 μg of hen egg albumin (OVA; Sigma) mixed with 100 mU of sialidase from Clostridium perfringens (Cp-NA, Sigma or Calbiochem), Salmonella typhimurium (St-NA; Sigma) or Arthrobacter urefaciens (Calbiochem).

In separate studies, protein antigens were administered to mice intranasally along with 5 μg anti-CD43 monoclonal antibody or 5 μg anti-CD43 monoclonal antibody and 30 mU of sialidase.

Collection of Serum and Mucosal Secretion

Serum samples were collected by tail-vein bleeding. Fecal pellets were dissolved in PBS containing 0.1% sodium azide (100 mg fecal pellet per 1 ml PBS/sodium azide). Vaginal washes were collected by gently flushing the vaginal canal with 150 μl of sterile PBS under anesthesia. Nasal washes were obtained by flushing the nasopharyngeal cavity with 100 μl of sterile PBS. Saliva was collected after intraperitoneal injection of 0.1 mg of pilocarpine (Sigma) in sterile PBS. After collection, nasal and vaginal washes and saliva samples were centrifuged for 5-10 min at 10,000 rpm and supernatants collected and stored at −70 24° C. until used.

Isolation of Cells from Mucosal and Systemic Compartments

Spleen cells were analyzed to evaluate the immune response in the systemic compartment. The immunity in the mucosal compartment was determined by analyzing Peyer's patches (PP) and intestinal lamina propria cells for the immune response at the intestinal level and lower respiratory tract cells for the immune response in the nasopharyngeal associated lymphoid tissue (NALT). Spleens were aseptically removed and teased through wire mesh to isolate cells. After washing, red blood cells were removed from the spleen mononuclear cell suspension by incubation 5 min. in 0.84% NH4Cl at 4° C. Peyer's patches were excised and cells were isolated by teasing through wire mesh.

For the isolation of small intestinal lamina propria lymphocytes (LPL), Peyer's patch-free intestine fragments 10 were incubated in 1 mM EDTA in PBS for 30 min. under agitation followed by collagenase treatment of tissue for 1 hour. Lamina propria lymphocytes were further purified from contaminating epithelial cells by discontinuous Percoll's gradient. Lower respiratory tract (lung) associated lymphoid cells were obtained by digesting the tissue with collagenase and purified by Percoll gradient.

Whole cell populations from each tissue were used for the analysis of antigen-specific Ab immunospot forming cells. To determine the nature of T helper cells involved in adjuvant activity of hemolysin, CD4₊ T cells were purified by negative MACS sorting.

Evaluation of Antigen-specific Ab Titers and Frequency of Antigen-specific Ab Secreting Cells

An ELISA assay was used to determine titers of mucosal S-IgA Abs and serum immunoglobulin isotype and subclass titers. The number of antigen-specific immunospot forming cells in each tissue was determined by ELISPOT assay.

Antigen-specific T Cells Responses

For antigen-specific T cell responses, CD4⁺ T cells were culture in the presence of 20% feeder cells obtained by irradiation (3000 rads) of T cell depleted spleen cells. Cells were stimulated with soluble OVA (500 μg/ml) or bead-coated TT (ratio 10 beads per cells) and incubated for 5-6 days at 37° C. in 5% CO₂ atmosphere. Antigen-specific proliferative responses were determined by measuring the [methyl-³H]-thymidine incorporation during the last 18 hours of culture. Culture supernatants were also collected and the nature of T helper cytokine secreted was determined by cytokine-specific ELISA assay.

It is to be understood that the instant invention does not relate to conjugated proteins such as the sialidases from influenza viruses, but that the administration of sialidases as taught herein relates to administration of a composition containing, as an antigen, vaccine and as an adjuvant, at least one sialidase wherein the sialidase and the antigen to which antibodies are sought to be raised are not present as conjugated proteins.

It is further to be understood that the particular value of the invention relates to use with vaccines wherein either the protal of entry or the site of reproduction of the organism responsible for disease production is the mucosa.

It is, of course, understood that any oral ingestion of a composition contacts the mucosa, since the entire gastro-intestinal tract is lined with mucosa.

What we claim is:

1. A composition of matter comprising, as active agents, an adjuvant-effective amount of at least one sialidase and (2) an unrelated antigenic vaccine which gives rise to antibodies against an organism whose protal of entry, site of reproduction or site of disease causation is the mucosa, in a pharmaceutically acceptable carrier.

2. A composition of claim 1 wherein the carrier is a solid support.

3. A composition of claim 1 which is a liquid.

4. A composition of claim 1 which is a powder.

5. A composition of claim 1 which is in capsule or tablet form.

6. A composition of claim 1 containing, additionally, anti-CD43 antibodies.

7. A composition of claim 1 which is a gel, a cream or a suppository.

8. A method of enhancing immune response in mammal by administration to the mucosa of an immune enhancing effective amount of sialidases in a pharmaceutically accepted carrier in conjunction with a antigenic vaccine.

9. A method of claim 8 wherein sialidase is administered in the form of drops or a spray.

10. A method of claim 8 wherein sialidase is administered in powder form.

11. A method of claim 8 wherein sialidase is administered on a solid support.

12. A method of claim 8 wherein sialidase is administered orally.

13. A method of claim 8 wherein sialidase is administered in animal feed.

14. A method of claim 8 wherein sialidase is administered in conjunction with an anti-CD4 antibody.

15. A method of claim 14 wherein the anti-CD4 antibody is an anti-CD43 antibody.
